(19) United States
(12) Patent Application Publication
(10) Pub. No.: US 2026/0224362 A1
(43) Pub. Date: Aug. 6, 2026

(54) CAPSULE EXTENSION TO MINIMIZE LEAFLET PROTRUSION

(71) Applicant: Medtronic, Inc., Minneapolis, MN (US)

(72) Inventor: Christopher MCCABE, Oranmore (IE)

(21) Appl. No.: 19/147,038

(22) PCT Filed: Jan. 12, 2024
(86) PCT No.: PCT/IB2024/050346

§ 371 Date: Jul. 10, 2025

Related U.S. Application Data
(60) Provisional application No. 63/481,807, filed on Jan. 27, 2023.

(51) Int. Cl. A61F 2/24; A61F 2/962; A61F 2/966

(52) U.S. Cl. CPC A61F 2/2436; A61F 2002/9623; A61F 2002/9665

(57) ABSTRACT

A delivery assembly for percutaneously delivering a transcatheter heart valve prosthesis into a vasculature of a patient includes a capsule and a capsule extension. The capsule is configured to constrain the transcatheter heart valve prosthesis therein. The capsule extension includes a mesh layer coupled to the capsule, reaching arms having a first end and a second end, the second end being coupled to the mesh layer, and a blocking arm coupled the first ends of the reaching arms. The blocking arm is configured to extend circumferentially around at least a portion of the transcatheter heart valve prosthesis to minimize leaflet protrusion through cells of a frame of the transcatheter heart valve prosthesis.

### CROSS-REFERENCE TO RELATED APPLICATION

This application claims the benefit of U.S. Provisional Patent Application Ser. No. 63/481,807, filed Jan. 27, 2023, the entire content of which is incorporated herein by reference.

### FIELD OF THE INVENTION

The present invention relates to a capsule extension that minimizes leaflet protrusion and leaflet damage during the loading/recapture of a transcatheter heart valve prosthesis into a delivery catheter and deployment of the transcatheter heart valve prosthesis.

### BACKGROUND

Diseases associated with heart valves, such as those caused by damage or a defect, can include stenosis and valvular insufficiency or regurgitation. For example, valvular stenosis causes the valve to become narrowed and hardened which can prevent blood flow to a downstream heart chamber from occurring at the proper flow rate and may cause the heart to work harder to pump the blood through the diseased valve. Valvular insufficiency or regurgitation occurs when the valve does not close completely, allowing blood to flow backwards, thereby causing the heart to be less efficient. A diseased or damaged valve, which can be congenital, age-related, drug-induced, or in some instances, caused by infection, can result in an enlarged, thickened heart that loses elasticity and efficiency. Some symptoms of heart valve diseases can include weakness, shortness of breath, dizziness, fainting, palpitations, anemia and edema, and blood clots which can increase the likelihood of stroke or pulmonary embolism. Symptoms can often be severe enough to be debilitating and/or life threatening.

Heart valve prostheses have been developed for repair and replacement of diseased and/or damaged heart valves. Such heart valve prostheses can be percutaneously delivered and deployed at the site of the diseased heart valve through catheter-based delivery systems. Such heart valve prostheses generally include a frame or stent and a prosthetic valve mounted within the frame. Such heart valve prostheses are delivered in a radially compressed or crimped configuration so that the heart valve prosthesis can be advanced through the patient's vasculature. Once positioned at the treatment site, the heart valve prosthesis is expanded to engage tissue at the diseased heart valve region to, for instance, hold the heart valve prosthesis in position.

In some circumstances, when radially compressing/crimping a heart valve prosthesis into delivery catheter, recapturing a heart valve prosthesis into a delivery catheter after at least partial deployment thereof, and/or during deployment of heart valve prosthesis from a delivery catheter, leaflets of the prosthetic heart valve may get trapped/pinched between struts of the frame as the frame is radially compressed/crimped into the radially compressed configuration or as the frame is radially expanded to the radially expanded configuration. This may cause damage to the leaflets and affect performance and longevity of the heart valve prosthesis in vivo. The present disclosure relates to improvements in delivery catheters to minimize or prevent pinching of the leaflets of the heart valve prosthesis during loading, recapture, and/or deployment thereof.

### BRIEF SUMMARY OF THE INVENTION

In accordance with a first example hereof, a delivery assembly for percutaneously delivering a transcatheter heart valve prosthesis into a vasculature of a patient is disclosed. The delivery assembly comprises a capsule configured to constrain the transcatheter heart valve prosthesis therein and a capsule extension. The capsule extension includes a mesh layer coupled to the capsule, reaching arms having a first end and a second end, the first end being coupled to the mesh layer, and a blocking arm coupled to the second ends of the reaching arms. The blocking arm is configured to extend circumferentially around at least a portion of the transcatheter heart valve prosthesis to minimize leaflet protrusion through cells of a frame of the transcatheter heart valve prosthesis.

In a second example, in the delivery assembly according to any of the previous or subsequent examples herein, the blocking arm is a blocking ring configured to extend circumferentially around the entire circumference the transcatheter heart valve prosthesis.

In a third example, in the delivery assembly according to any of the previous or subsequent examples herein, the reaching arms comprise six reaching arms, wherein the blocking arm comprises three blocking arms, and wherein each blocking arm is coupled to the second ends of a corresponding adjacent pair of the reaching arms.

In a fourth example, in the delivery assembly according to any of the previous or subsequent examples herein, the mesh layer includes a first end coupled to a distal end of the capsule.

In a fifth example, in the delivery assembly according to any of the previous or subsequent examples herein, the delivery assembly includes a delivery configuration in which the capsule extension extends in a first direction from the first end of the mesh layer, folds inwardly at a fold, and extends from the fold in a second direction opposite the first direction.

In a sixth example, in the delivery assembly according to any of the previous or subsequent examples herein, the first direction is a proximal direction and the second direction is a distal direction.

In a seventh example, in the delivery assembly according to any of the previous or subsequent examples herein, the first direction is a distal direction and the second direction is a proximal direction.

In an eighth example, in the delivery assembly according to any of the previous or subsequent examples herein, further comprising a transcatheter heart valve prosthesis, the transcatheter heart valve prosthesis including a frame and a valve structure coupled to the frame, wherein the valve structure includes a plurality of leaflets, and wherein the frame includes an access cell larger than other cells of the frame.

In a ninth example, in the delivery assembly according to any of the previous or subsequent examples herein, in a delivery configuration, the transcatheter heart valve prosthesis is disposed within the capsule and the capsule extension is disposed between the transcatheter heart valve prosthesis and the capsule.

In a tenth example, in the delivery assembly according to any of the previous or subsequent examples herein, in a deployed configuration of the capsule extension, the capsule extension extends distally from a distal end of the capsule, and the blocking arm extends circumferentially around the transcatheter heart valve prosthesis and across the access cell.

In an eleventh example, in the delivery assembly according to any of the previous or subsequent examples herein, in the deployed configuration of the capsule extension, the transcatheter heart valve prosthesis is partially deployed from the capsule.

In a twelfth example, in the delivery assembly according to any of the previous or subsequent examples herein, in the deployed configuration of the capsule extension, the transcatheter heart valve prosthesis is at 0% to 70% deployment, or 20% to 50% deployment, or 30% to 40% deployment.

In a thirteenth example, a method of delivering and deploying a transcatheter heart valve prosthesis to a treatment site within a vasculature of a patient is disclosed. The method comprises delivering the transcatheter heart valve prosthesis to the treatment site with the transcatheter heart valve prosthesis radially compressed within a capsule of a delivery device, the transcatheter heart valve prosthesis including a frame and a valve structure coupled to the frame, wherein the valve structure includes a plurality of leaflets, wherein the frame includes an access cell larger than other cells of the frame, and wherein the delivery device further includes a capsule extension coupled to the capsule and disposed between the transcatheter heart valve prosthesis and the capsule in a delivery configuration. The method further comprises proximally retracting the capsule to release the transcatheter heart valve prosthesis from the capsule and transitioning the capsule extension to a deployed configuration of the capsule extension, wherein in the deployed configuration of the capsule extension, the capsule extension extends distally from a distal end of the capsule and surrounds a portion of the transcatheter heart valve prosthesis, and a blocking arm of the capsule extension extends circumferentially around at least a portion of the transcatheter heart valve prosthesis and across the access cell to minimize leaflet protrusion through the access cell. The method further comprises determining whether to fully deploy the transcatheter heart valve prosthesis or to recapture the transcatheter heart valve prosthesis, wherein if it is determined to recapture the transcatheter heart valve prosthesis, distally advancing the capsule to recapture the transcatheter heart valve prosthesis, and wherein if it is determined to deploy the transcatheter heart valve prosthesis, proximally retracting the capsule further to fully deploy the transcatheter heart valve prosthesis.

In a fourteenth example, in a method according to any of the previous or subsequent examples herein, the blocking arm is a blocking ring, and wherein in the deployed configuration of the capsule extension, the blocking ring extends circumferentially around the entire circumference the transcatheter heart valve prosthesis.

In a fifteenth example, in a method according to any of the previous or subsequent examples herein, the capsule extension further includes a mesh layer coupled to the distal end of the capsule, reaching arms coupled at a first end thereof to the mesh layer and at a second end thereof to the blocking arm.

In a sixteenth example, in a method according to any of the previous or subsequent examples herein, in the delivery configuration, the capsule extension extends in a first direction from the first end of the mesh layer, folds inwardly at a fold, and extends from the fold in a second direction opposite the first direction.

In a seventeenth example, in a method according to any of the previous or subsequent examples herein, the first direction is a proximal direction and the second direction is a distal direction.

In an eighteenth example, in a method according to any of the previous or subsequent examples herein, the first direction is a distal direction and the second direction is a proximal direction.

In a nineteenth example, in a method according to any of the previous or subsequent examples herein, in the deployed configuration of the capsule extension, the transcatheter heart valve prosthesis is partially deployed from the capsule.

In a twentieth example, in a method according to any of the previous or subsequent examples herein, in the deployed configuration of the capsule extension, the transcatheter heart valve prosthesis is at 0% to 70% deployment, or 20% to 50% deployment, or 30% to 40% deployment.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

The foregoing and other features and advantages of the present disclosure will be apparent from the following description of embodiments hereof as illustrated in the accompanying drawings. The accompanying drawings, which are incorporated herein and form a part of the specification, further serve to explain the principles of the present disclosure and to enable a person skilled in the pertinent art to make and use the embodiments of the present disclosure. The drawings may not be to scale.

FIG. 1A shows a side view of an example of a transcatheter heart valve prosthesis.

FIG. 1B shows an outflow view of the transcatheter heart valve prosthesis of FIG. 1A.

FIG. 2A is a schematic perspective view of a delivery assembly in a delivery configuration according to embodiments hereof.

FIG. 2B is a schematic perspective view of a delivery assembly of FIG. 2A in a deployed configuration according to embodiments hereof.

FIG. 3A is a schematic of a side cross-section of a distal portion of the delivery assembly of FIG. 2A in the delivery configuration, showing a capsule extension according to embodiments hereof.

FIG. 3B shows a distal view of FIG. 3A.

FIG. 3C is a schematic of a side cross-section of the capsule and capsule extension of FIG. 3A during deployment of the transcatheter heart valve prosthesis.

FIG. 3D shows a side view of the capsule and capsule extension of FIG. 3A with a transcatheter heart valve prosthesis partially deployed from the capsule and the capsule extension in a deployed configuration according to an embodiment hereof.

FIG. 4 shows a side view of the distal portion of the delivery assembly of FIG. 2A with a transcatheter heart valve prosthesis partially deployed from the capsule and the capsule extension according to another embodiment hereof.

FIG. 5A is a schematic of a side cross-section of a distal portion of the delivery assembly of FIG. 2A in the delivery configuration including a capsule extension according to another embodiment hereof.

FIG. 5B is a schematic of a side cross-section of the distal portion of the capsule and capsule extension of FIG. 5A transitioning from the delivery configuration to a deployed configuration.

FIG. 5C is a schematic of a side cross-section of the distal portion of the capsule and capsule extension of FIG. 5A with the capsule extension in the deployed configuration.

### DETAILED DESCRIPTION

It should be understood that various embodiments disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single device or component for purposes of clarity, it should be understood that the techniques of this disclosure may be performed by a combination of devices or components associated with, for example, a delivery device. The following detailed description is merely exemplary in nature and is not intended to limit the invention of the application and uses of the invention. Furthermore, there is no intention to be bound by any expressed or implied theory presented in the preceding field of the invention, background, summary or the following detailed description.

As used in this specification, the singular forms “a”, “an” and “the” specifically also encompass the plural forms of the terms to which they refer, unless the content clearly dictates otherwise. The term “about” is used herein to mean approximately, in the region of, roughly, or around. When the term “about” is used in conjunction with a numerical range, it modifies that range by extending the boundaries above and below the numerical values set forth. It should be understood that use of the term “about” also includes the specifically recited number of value. When the term “approximately” or “approximately equivalent” is used in conjunction with a numerical range, the numerical range includes the specifically recited number of value and tolerance of 5%, meaning the upper and lower boundaries of the range is extended 5% above and 5% below the numerical value set forth.

The terms “proximal” and “distal” herein are used with reference to the clinician using the delivery devices. Therefore, “proximal” and “proximally” mean in the direction toward the clinician, and “distal” and “distally” mean in the direction away from the clinician.

Embodiments hereof relate to delivery assembly including a capsule having a capsule extension configured for use with a transcatheter heart valve prosthesis when loading the transcatheter heart valve prosthesis into the capsule for delivering the transcatheter heart valve prosthesis to a treatment site within a vasculature of a patient and deploying the transcatheter heart valve prosthesis at the treatment site. More particularly, the capsule extension is configured to prevent, or block, leaflets of the transcatheter heart valve prosthesis from protruding through cells or openings in a frame of the transcatheter heart valve prosthesis during loading of and deployment of the transcatheter heart valve prosthesis that may cause leaflet pinching and damage. The capsule extension includes at least one blocking arm or a blocking ring such that when the transcatheter heart valve prosthesis is being loaded into the capsule, and/or is being deployed from the capsule, and/or is being recaptured into the capsule, the at least one blocking arm or blocking ring prevents the leaflets from protruding through an access cell of the frame of the transcatheter heart valve prosthesis, thereby minimizing potential damage to the leaflets during loading, deployment, and/or recapture.

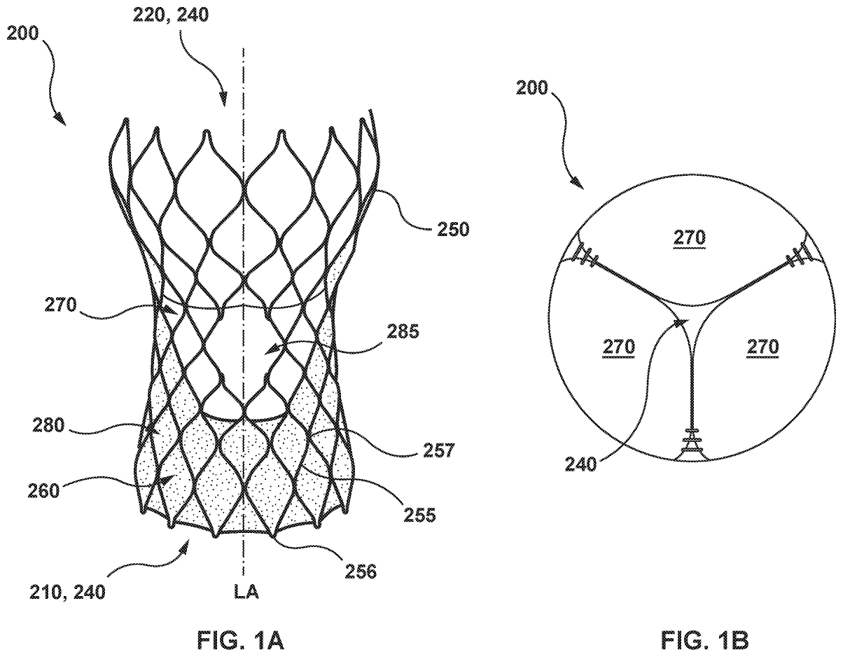

FIGS. 1A and 1B illustrate an example transcatheter heart valve prosthesis 200. The delivery assemblies described herein may be used with the transcatheter heart valve prosthesis 200 and/or other transcatheter heart valve prostheses. The transcatheter heart valve prosthesis 200 is illustrated herein in order to facilitate description of the present invention. The following description of the transcatheter heart valve prosthesis 200 is merely exemplary in nature and is not intended to limit the invention or the application and uses of the invention.

FIGS. 1A and 1B illustrate a side view and a top (outflow end) view, respectively, of the transcatheter heart valve prosthesis 200. The transcatheter heart valve prosthesis 200 includes a radially-expandable frame or stent 250 and a prosthetic valve 260. The frame 250 of the transcatheter heart valve prosthesis 200 supports the prosthetic valve 260 within the interior of the frame 250. In the example transcatheter heart valve prosthesis 200 shown in FIGS. 1A-1B, the frame 250 is self-expandable. However, this is not meant to be limiting, and the frame 250 can be balloon-expandable or mechanically expandable.

The prosthetic valve 260 includes at least one leaflet 270 disposed within and secured to the frame 250. In the embodiment shown in FIGS. 1A-1B, the prosthetic valve 260 includes exactly three leaflets 270, as shown in FIG. 1B. However, this is not meant to be limiting, as the prosthetic valve 260 may include more or fewer leaflets 270. The valve leaflets 270 open and close to regulate flow through the transcatheter heart valve prosthesis 200.

As shown in FIG. 1A, the transcatheter heart valve prosthesis 200 includes an inflow end 210 and an outflow end 220. The prosthetic leaflets 270 are attached to the frame 250 such that when pressure at the inflow end 210 exceeds pressure at the outflow end 220, the prosthetic leaflets 270 open to allow blood flow through the heart valve prosthesis from the inflow end 210 to the outflow end 220. When the pressure at the outflow end 220 exceeds pressure at the inflow end 210, the prosthetic leaflets 270 close to prevent blood flow from the outflow end 220 to the inflow end 210.

The frame 250 of the transcatheter heart valve prosthesis 200 further includes a plurality of struts 255 that are arranged to form a plurality of side openings or cells 280 arranged circumferentially around a longitudinal axis LA of the transcatheter heart valve prosthesis 200 and longitudinally to form a tubular structure defining a central lumen 240 of the transcatheter heart valve prosthesis 200. The struts 255 are defined herein as the elongated wire segments of the frame 250. Struts 255 come together to form crowns 256 or nodes node 257, as can be seen in FIG. 1A. The frame 250 is configured to secure the prosthetic valve 260 within the central lumen 240 of the frame 250 and to secure the transcatheter heart valve prosthesis 200 in place in the vasculature of the patient.

The frame 250 of the heart valve prosthesis 200 includes a plurality of cells 280 defined as the spaces between the plurality of crowns 256, the plurality of nodes 257 and the plurality of struts 255. In the example embodiment shown in FIG. 1A, the plurality of cells 280 may be diamond-shaped. In the example embodiment shown, the plurality of cells include a plurality of first cells 280 and access cells 285. In particular, the access cells are larger than the first cells 280. In the embodiment shown, there are exactly three access cells 285. However, this is not meant to be limiting, as the frame 250 of the transcatheter heart valve prosthesis 200 can include more or fewer access cells 285. The access cells 285 each have an enlarged area relative or compared to the first cells 280, as can be seen in FIG. 1A. Further, the access cells 285 may be located in other locations than the locations shown in FIG. 1A.

As explained above, a delivery assembly may be used to deliver the transcatheter heart valve prosthesis 200 in a crimped configuration and to deploy the transcatheter heart valve prosthesis 200 to an expanded configuration at the treatment site within the vasculature. When the heart valve prosthesis 200 is loaded into the delivery assembly or recaptured into the delivery assembly, as the frame 250 is radially compressed or crimped to the crimped configuration, the first cells 280 and the access cells 285 also reduce in size, potentially trapping or pinching the one or more of the leaflets 270 between the struts defining one or more of the first cells or access cells. Further, during the deployment process, expansion of the frame 250 of the transcatheter heart valve prosthesis 200 causes the area of the plurality of cells 280 of the frame 250 to increase in size. In some instances, one or more of the leaflets 270 of the prosthetic valve 260 may protrude through one of the cells 280/285 during the expansion process, which may cause the leaflets 270 to get pinched, or stuck, between the struts 255 of the frame 250 and sustain damage such as tearing. Leaflet protrusion becomes increasingly likely as the area of the cells increase. Therefore, it is more likely to occur at the access cells 285 than the first cells 280. Accordingly, embodiments of a delivery assembly of the present invention minimize the risk of leaflet protrusion, as described in more detail below.

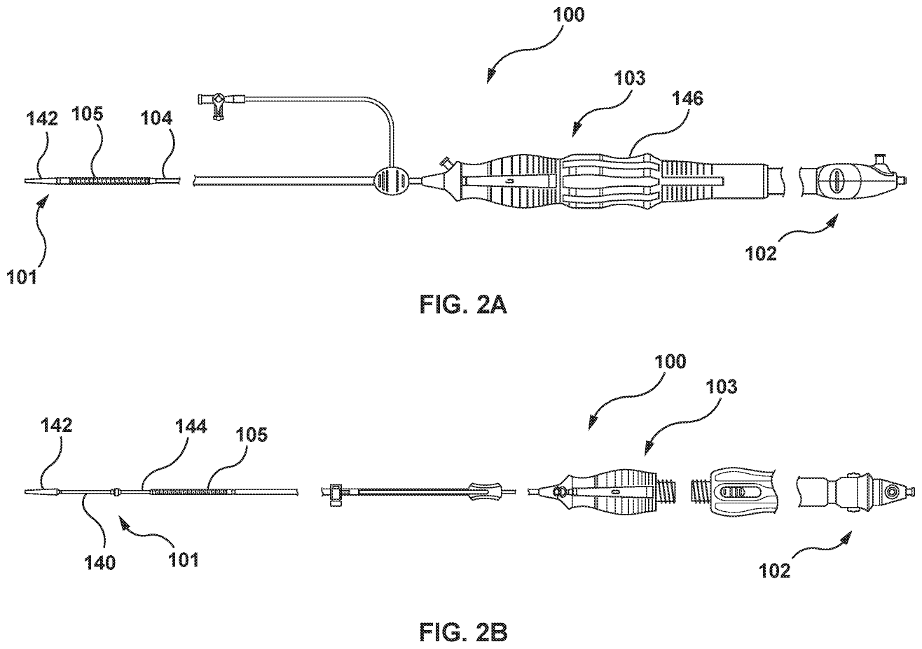

FIGS. 2A-2B show schematically side views of a delivery assembly 100 for delivering and deploying a transcatheter heart valve prosthesis according to embodiments hereof. One skilled in the art will realize that FIGS. 2A-2B illustrate one example of a delivery assembly and that components illustrated in FIGS. 2A-2B may be removed and/or additional components may be added. The delivery assembly 100 includes a distal end 101, a proximal end 102, and a handle 103. The handle 103 enables a clinician to manipulate a distal portion of the delivery assembly 100 and includes actuators for moving parts of the delivery system relative to other parts. In the delivery system 100, an outer shaft 104 is coupled to an actuator of the handle 103 for moving the outer shaft 103 relative to an inner shaft 140. A distal portion of the outer shaft 104, referred to as a capsule 105, is configured to surround a transcatheter heart valve prosthesis during delivery to the treatment site, e.g., a native heart valve and is retracted from the transcatheter heart valve prosthesis to expose the transcatheter heart valve prosthesis such that it self-expands. The inner shaft 140 is coupled to the handle 103 and movement of the handle 103 translates to movement of the inner shaft 140 and a distal tip 142 coupled to a distal end of the inner shaft 140. The inner shaft 140 and distal tip 142 may also be translated relative to the outer shaft 104 and the handle 103 via a tip retractor. In the embodiment shown, a middle member 144 is disposed between the inner shaft 140 and the outer shaft 104, and the middle member 144 includes a retainer or spindle attached to a distal portion thereof for receiving the paddles of the transcatheter heart valve prosthesis 200. The handle 103 includes an actuator 146 that when actuated moves the outer shaft 104 and the capsule 105 relative to the inner shaft 140, as shown in FIG. 2B. As known to those skilled in the art, when the delivery assembly 100 is in position such that the transcatheter heart valve prosthesis 200 is at the desired position at the treatment site in the patient's vasculature, the actuator 146 is actuated to move the capsule 105 relative to the inner shaft 140 and the transcatheter heart valve prosthesis 200 disposed between the inner shaft 140 and the capsule 105, thereby enabling the transcatheter heart valve prosthesis 200 to deploy via self-expansion at the treatment site, as shown in FIG. 2B (without showing the transcatheter heart valve prosthesis 200).

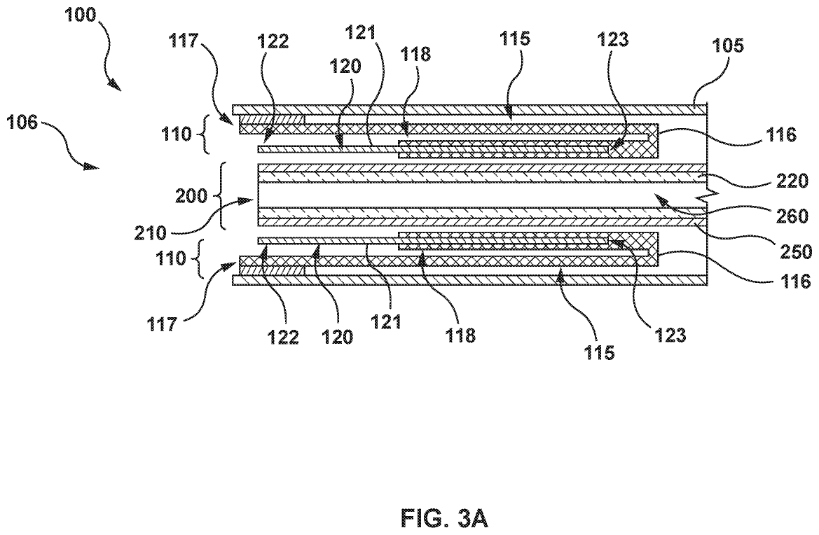

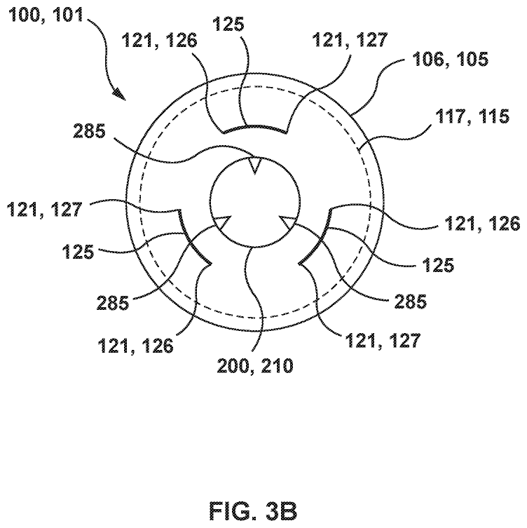

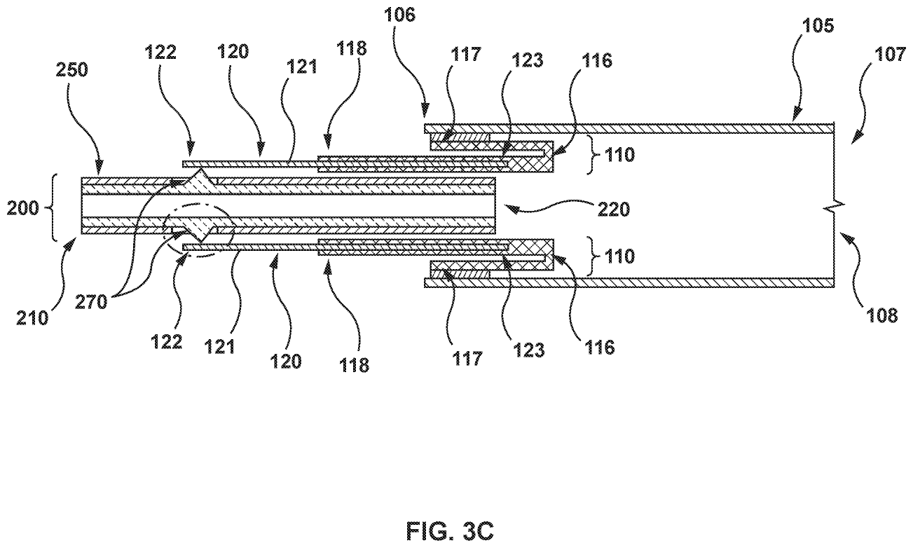

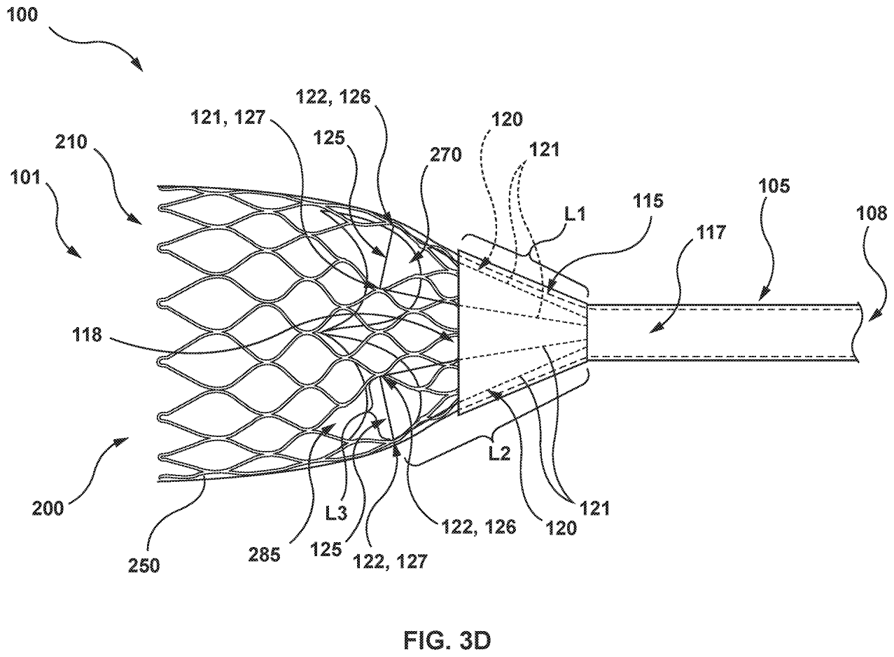

As explained above, in the delivery configuration, the capsule 105 of the delivery assembly 100 contains the transcatheter heart valve prosthesis 200 therein for delivery and deployment of the transcatheter heart valve prosthesis 200 to a desired treatment site. FIGS. 3A-3C show an embodiment of a capsule 105 including a mechanism to minimize leaflet protrusion, according to embodiments hereof. One skilled in the art will realize that FIGS. 3A-3C illustrate one example of the capsule 105 and that components illustrated in FIGS. 3A-3C may be removed and/or additional components may be added. FIGS. 3A and 3C show side cross-sections of the capsule 105 with the transcatheter heart valve prosthesis 200 disposed therein. The inner shaft 140 is not shown in FIGS. 3A and 3C for clarity. As shown, the capsule 105 includes a capsule extension 110, as will be described in more detail below.

The capsule 105 is a longitudinal tube having a distal end 106, a proximal end 107, and a central lumen 108 extending the entire longitudinal length of the capsule 105, extending from the distal end 106 to the proximal end 107. The central lumen 108 of the capsule 105 is sized and shaped to contain the capsule extension 110 and the transcatheter heart valve prosthesis 200 in the crimped configuration.

In the embodiment shown in FIGS. 3A-3D, the capsule extension 110 includes a mesh layer 115 and three blockers 120 (only two blockers 120 are shown in FIGS. 3A, 3C, and 3D). However, the use of three blockers 120 in this embodiment matches the three large access cells 285 in the frame 250 of the transcatheter heart valve prosthesis 200, and is not mean to be limiting. In other embodiments, more or fewer blockers may be utilized. In the embodiment of FIGS. 3A-3D, each blocker 120 includes two reaching arms 121 and a blocking arm 125 (shown in FIG. 3D). However, this is not meant to be limiting and each blocker 120 may include more reaching arms and more blocking arms. The mesh layer 115 is a tubular-shaped mesh component that includes a first end 117, a second end 118, and a lumen extended therethrough. The first end 117 of the mesh layer 115 is coupled to an interior circumference of the distal end 106 of the capsule 105, as shown in FIGS. 3A and 3C, such as by adhesive bonding, thermal bonding, or other attachment mechanisms. In an embodiment, the entire circumference of the first end 117 of the mesh layer 115 is bonded to the entire circumference of the interior surface of the capsule 105, however, this is not meant to be limiting. In the embodiment of FIGS. 3A-3D, when in the loaded or delivery configuration as shown in FIG. 3A, the mesh layer 115 extends from the distal end 107 of the capsule 105 proximally within the central lumen 108 of the capsule 105 and folds radially inward at a fold 116. The mesh layer 115 provides stability to the capsule extension 110 and creates a secure connection to the capsule 105. The mesh layer 115 may be formed of bioinert polymer e.g. polyurethane or LDPE (low-density polyethylene), or other suitable materials.

In the embodiment shown and as explained above, the capsule extension 110 further includes the three blockers 120, with each blocker 120 including two reaching arms 121. Each reaching arm 121 includes a first end 123 and a second end 122. The first end 123 of each of the reaching arms 121 is bonded to or embedded within the second end 118 of the mesh layer 115, and each reaching arms extends therefrom such that the second end 122 of each reaching arm 121 is distal of the second end 118 of the mesh layer, as can be seen in FIGS. 3A, 3C, and 3D. The first ends 123 of the reaching arms 120 can be bonded to an interior surface of the second end 118 of the mesh layer 115, or bonded to an exterior surface of the second end 118 of the mesh layer 115, or embedded within the second end 118 of the mesh layer 115. The overlap between the first ends 123 of the reaching arms 121 and the second end 118 of the mesh layer 115 can range from about 15 mm-20 mm. The reaching arms 121 may be longitudinally straight strands or wires made of polymer, polyurethane, or similar materials.

In the embodiment shown in FIGS. 3A-3D, each blocker 120 further includes a blocking arm 125. Each of blocking arm 125 includes a first end 126 and a second end 127. Each of the blocking arms 125 connects the second ends 123 of two directly adjacent reaching arms 121. As such, each the blocking arms 125 can be described as extending in a generally circumferential direction. The first end 126 of the blocking arm 125 couples to the second end 122 of a first reaching arm 121 and the second end 127 of the blocking arm 125 couples to the second end 122 of a second reaching arm 121 directly adjacent to the first reaching arm 121, creating a substantially triangular shape between the two adjacent reaching arms 121 and the blocking arm 125, as best shown in FIG. 3D. Each blocking arm 125 is configured extend across one of the access cells 285 to block one of the leaflets 270 of the transcatheter heart valve prosthesis 200 from protruding through the access cell 285 during deployment of the transcatheter heart valve prosthesis 200. The blocking arms 125 may be made of polymer, polyurethane, or similar materials.

The capsule 105 and capsule extension 110 described herein includes a delivery configuration, as shown in FIGS. 3A-3B, and a deployed configuration, as shown in FIGS. 3C-3D. In the delivery configuration, the entire capsule extension 110 is loaded within the central lumen 108 of the capsule 105, as shown in in FIG. 3A. The transcatheter heart valve prosthesis 200 is loaded within the central lumen 108 of the capsule 105 in the crimped configuration and is disposed radially inward in relation to the capsule extension 110. In the embodiment shown in FIG. 3A, the inflow end 210 of the frame 250 is disposed adjacent the distal end 106 of the capsule 105 and the outflow end 220 of the frame 250 is disposed near the proximal end 107 of the capsule 105. However, this is not meant to be limiting and the opposite orientation may be used depending on the native valve being replaced and the route to be taken by the delivery assembly 100 to reach the native valve. As shown in FIG. 3A, when the delivery assembly 100 is in the delivery configuration, the first end 117 of the mesh layer 115 is coupled to the interior surface of the distal end 106 of the capsule 105 and the mesh layer 115 extends proximally towards the proximal end 107 of the capsule 105 until it reaches the fold 116. At the fold 116, the mesh layer 115 folds radially inward, towards the crimped transcatheter heart valve prosthesis 200, and extends back distally towards the distal end 106 of the capsule 105. In this configuration, the second end 118 of the mesh layer 115 terminates proximal to the first end 117 of the mesh layer 115 and is disposed distal to the fold 116. Additionally, the second end 118 of the mesh layer 115 is disposed radially inward in relation to the first end 117 of the mesh layer 115. In this embodiment, the first ends 123 of the reaching arms 121 are bonded to or embedded within the second end 118 of the mesh layer 115 and the reaching arms 121 extend distally therefrom. In the delivery configuration, the second ends 122 of the reaching arms 121 terminate adjacent the distal end 106 of the delivery capsule 105 and are disposed radially inward in relation to the first end 117 of the mesh layer 115 and the capsule 105, as shown in FIG. 3A. The reaching arms 121 are disposed radially inward relative to the first end 117 of the mesh layer 115 and the capsule 105.

FIG. 3B shows a cross-section view of the capsule 105 in the delivery configuration. As discussed above, the frame 250 of the transcatheter heart valve prosthesis 200 includes exactly three access cells 285. The exactly three access cells 285 are equidistantly spaced apart from one another in a radial direction around the circumference of the frame 250. In the delivery configuration, the transcatheter heart valve prosthesis 200 is in the crimped configuration. As can be seen in FIG. 3B, the capsule extension 110 includes exactly three blocking arms 125 corresponding to the exactly three access cells 285 of the frame 250, which will be discussed in further detail below. The first and second ends 126, 127 of the blocking arms 125 are coupled to the second ends 122 of exactly six reaching arms 121 (not shown). In the delivery configuration, the three blocking arms 125 and the six reaching arms 121 (not shown) of the capsule extension 110 are disposed radially outward from the transcatheter heart valve prosthesis 200. In the delivery configuration, the blocking arms 125 may be disposed in-line with or adjacent the distal end 106 of the delivery capsule 105, where the second ends 122 of the reaching arms 121 terminate. In the delivery configuration, the three blocking arms 125 are circumferentially aligned with the positions of the three access cells 285 of the frame 250 such that as the transcatheter heart valve prosthesis 200 is deployed from the delivery assembly 100, the blocking arms 125 may align with and extend over a portion of the access cells 285 of the transcatheter heart valve prosthesis 200, which will be described in further detail below.

As can be seen in FIG. 3B, the first end 117 of the mesh layer 115 is bonded to the interior surface of the distal end 106 of the capsule 105 at bond 119 and is disposed radially inward from the delivery capsule 105. In FIG. 3B, the first end 117 of the mesh layer 115 is shown spaced apart from the capsule 105, but this is merely to show distinction between the mesh layer 115 and the capsule 105 for clarity purposes. As shown in FIG. 3B, the three blocking arms 125 of the capsule extension 110 are disposed radially inward from the first end 117 of the mesh layer 115 and the capsule 105. When the delivery assembly 100 is in the delivery configuration, the transcatheter heart valve prosthesis 200 is in the crimped configuration and is disposed radially inward from the blocking arms 125 of the capsule extension 110.

To transition the delivery assembly 100 from the delivery configuration to the deployed configuration, the capsule 105 is retracted proximally in order to release, or uncover, the transcatheter heart valve prosthesis 200 and the capsule extension 110. In other words, as the distal end 106 of the capsule 105 is retracted proximally, the capsule extension 110 and the transcatheter heart valve prosthesis 200 remain in place such that they exit the central lumen 108 of the capsule 105 through the distal end 106 of the capsule 105. As the capsule 105 is slowly retracted proximally, the inflow end 210 of the transcatheter heart valve prosthesis 200 may begin to self-expand in the vasculature, as it is no longer enclosed by the capsule 105. As the capsule 105 translates proximally and uncovers, or releases, the transcatheter heart valve prosthesis 200 and the capsule extension 110, the blocking arms 125 align with the respective access cells 285 of the frame 250 such that each blocking arm 125 may prevent a respective valve leaflet 270 from protruding through the respective access cell 285 of the frame 250 when the transcatheter heart valve prosthesis 200 begins to expand within the vasculature of the patient. As the capsule 105 is retracted proximally, the first end 117 of the mesh layer 115 is simultaneously retracted proximally. As the distal end 106 of the capsule 105 and the first end 117 of the mesh layer 115 are retracted proximally, the fold 116 of the mesh layer 115 translates distally within the central lumen 108 relative to the capsule 105 such that the fold 116 moves towards the distal end 106 of the capsule 105, as shown in FIG. 3C as compared to FIG. 3A. When the fold 116 reaches the first end 117 of the mesh layer 115 where the first end 117 is coupled to the interior surface of the capsule 105, the capsule extension 110 is fully deployed. In other words, referring to FIG. 3C, when the fold 116 reaches the proximal end of the bond 119, the capsule extension 110 cannot extend any further distally from the capsule 105. The first end 117 of the mesh layer 115 is bonded to the distal end 106 of the capsule 105 and does not move or translate relative to the capsule 105 during the transition to the deployed configuration. When the capsule extension 110 is in the deployed configuration, the blocking arms 125, the reaching arms 121, and the second end 118 of the mesh layer 115 are disposed distal to the distal end 106 of the capsule 105.

As the capsule 105 continues to be retracted proximally from the transcatheter heart valve prosthesis 200, the capsule extension 110 begins to move with the capsule 105 because, as described above, the fold 116 has reached the bond 119. As the capsule 105 and the capsule extension 110 continue to be retracted proximally, the reaching arms 121 and the blocking arms 125 will also retract proximally relative to the with the transcatheter heart valve prosthesis 200. As a predetermined stage of the deployment, the blocking arms 125 will extend across corresponding access cells 285 of the frame 250 of the transcatheter heart valve prosthesis. In an embodiment, the blocking arms 125 extend across a center portion of the corresponding access cells 285, which is defined to mean the substantially center portion of the access cell 285 where the width of the access cell 285 is the greatest. As shown in FIGS. 3C and 3D, the blocking arms 125 extend over the center portion of the corresponding access cells 285 such that each blocking arm 125 may block a leaflet 270 of the transcatheter heart valve prosthesis 200 from protruding through the access cell 285 of the frame 250. In an embodiment, the blockings arm 125 are configured to extend over the corresponding access cells 285 of the frame 250 when the transcatheter heart valve prosthesis 200 reaches approximately 70% deployment. At approximately 70% deployment, a user will be able to evaluate the function of the transcatheter heart valve prosthesis 200, and if functioning properly, recapture and re-deployment of the transcatheter heart valve prosthesis 200 is unnecessary. In other embodiments, the capsule extension 110 can be configured such that the blocking arms 125 extend across the center portion of the corresponding access cells 285 at other stages of the deployment. For example, and not by way of limitation, the capsule extension 110 can be configured such that the blocking arms 125 extend across the center portion of the corresponding access cells 285 from 0% to approximately 70% deployment, or at 20% to 50% deployment, or 30% to 40% deployment. The capsule extension 110 can be configured such that the blocking arms 125 extend across the center portion of the corresponding access cells 285 at different stages of deployment by, for example, and not by way of limitation, longitudinal location of the access cells 285, length of the mesh layer 115, and length of the reaching arms 121.

Once it has been determined that the transcatheter heart valve prosthesis 200 can be fully deployed, the capsule 105 continues to be proximally retracted, which proximally retracts the capsule extension 110 until the capsule 105 and the capsule extension 110 (including the mesh layer 115, the reaching arms 121, and the blocking arms 125) no longer surround any part of the transcatheter heart valve prosthesis 200. In particular, in the embodiment shown, the capsule 105 is retracted until the capsule 105 and the capsule extension 110 are retracted past the outflow end 220 of the transcatheter heart valve prosthesis 200 to enable the transcatheter heart valve prosthesis 200 to fully expand in the vasculature. Once the transcatheter heart valve prosthesis 200 is fully deployed, the delivery assembly 100 may be retracted proximally until it is to be removed from the vasculature of the patient.

As shown in FIG. 3D, a length LI of the mesh layer 115 is such that when the capsule extension 110 is fully deployed, the mesh layer 115 does not overlap with the access cells 285 of the frame 250 of the transcatheter heart valve prosthesis 200 during delivery to prevent occluding or obstructing blood flow during deployment. Thus, the length LI of the mesh layer 115 may depend on the size of the transcatheter heart valve prosthesis 200 and the placement of the access cells 285 within the frame 250. The length LI of the mesh layer 115 can range from approximately 35 mm-40 mm. Similarly, a length L2 of the reaching arms 121 may depend on the size of the transcatheter heart valve prosthesis 200, the length of the mesh layer 115, and the location of the access cells 285. The length L2 of the reaching arms 121 is such that the second ends 122 of the reaching arms 120 may reach a center portion of the access cell 285 of the frame 250 when the capsule extension 110 reaches full deployment and the transcatheter heart valve prosthesis 200 reaches the desired deployment, such as approximately 70% deployment noted above. The length L2 of the reaching arms 121 can range from approximately 30 mm-35 mm. Additionally, a length L3 of each blocking arm 125 may depend on the size of the access cells 285 of the frame 250. The length L3 of the blocking arm 125 may be such that it may reach, or extend over, and entire width (circumferential direction) of the center portion of the access cell 285 of the frame 250. The length L3 of each blocking arm 125 can range from approximately 5 mm-9 mm.

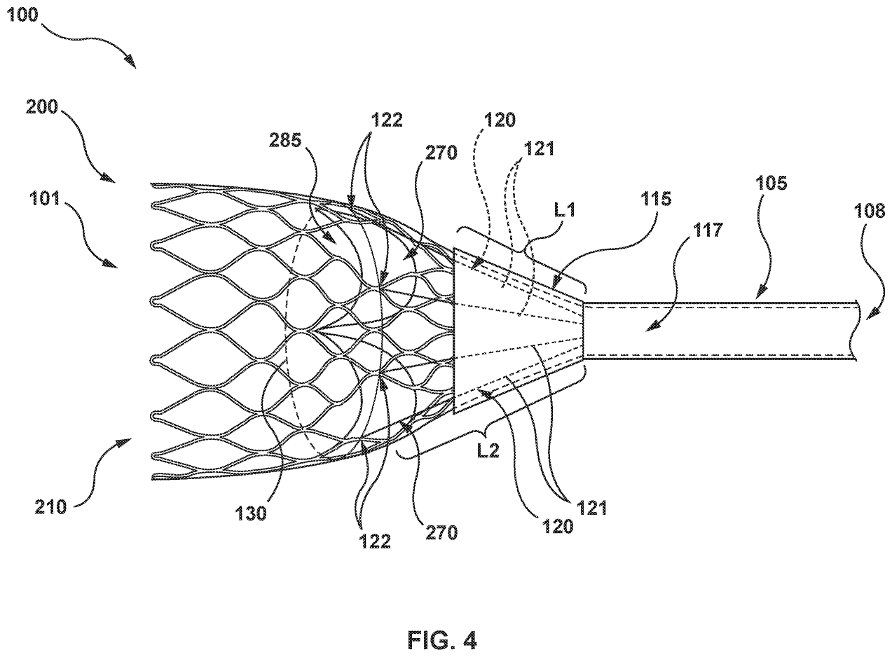

In some embodiments, the frame 250 of the transcatheter heart valve prosthesis 200 may only include one access cell 285. In such an embodiment, the capsule extension 110 would include exactly one blocker 120 including two directly adjacent reaching arms 121 and exactly one blocking arm 125 extending therebetween. In other embodiments, the frame 250 of the transcatheter heart valve prosthesis 200 may include exactly two access cells 285. In that embodiment, the capsule extension 110 would include exactly two blockers 120 including four reaching arms 121 and exactly two blocking arms 125. Although FIGS. 3 and 4 are only able to show two access cells 285, the frame 250 of the transcatheter heart valve prosthesis 200 described herein includes exactly three access cells 285 such that the capsule extension 110 described herein includes exactly six reaching arms 120 and exactly three blocking arms 125. In further embodiments, the transcatheter heart valve prosthesis 200 may include more than three access cells 285. For each access cell 285 of the frame 250 of the transcatheter heart valve prosthesis 200, the capsule extension 110 may include one blocker 120 with exactly two directly adjacent reaching arms 121 and exactly one blocking arm 125.

In another embodiment, the blocking arms 125 of the capsule extension 110 can be replaced with a blocking ring 130, as shown in FIG. 4. The blocking ring 130 is a circumferential ring that couples to the second ends 122 of the reaching arms 121 of the capsule extension 110. Although FIG. 4 shows two access cells 285 and four reaching arms 121, a third access cell 285 is not shown. Further, as explained above, more or fewer access cells 285 may be included. Similarly, as opposed to the embodiment described above with individual blocking arms 125 corresponding to each access cell 285, in the embodiment with the blocking ring 130, the capsule extension 110 need only include reaching arms 121 to connect the mesh layer 115 to the blocking ring 130. For example, and not by way of limitation, two reaching arms 121 may be attached to or embedded within the mesh layer 115 and coupled to the blocking ring 130 at 180 degrees apart from each other, or three reaching arms 121 may be attached to or embedded within the mesh layer 115 and coupled to the blocking ring 130 at 120 degrees apart from each other, or four reaching arms 121 may be attached to or embedded within the mesh layer 115 and coupled to the blocking ring 130 at 90 degrees apart from each other, or any number of suitable reaching arms 120 may be attached to or embedded within the mesh layer 115 and coupled to the blocking ring 130 and distributed around a central axis thereof at an appropriate spacing. The blocking ring 130 may be formed from polymer, polyurethane, or similar.

The blocking ring 130 is sized to extend circumferentially around the transcatheter heart valve prosthesis 200 when it is being deployed within the vasculature of a patient. More particularly, the blocking ring 130 is sized to extend circumferentially around the transcatheter heart valve prosthesis 200 where the center portion of the access cells 285 are located on the frame 250 of the transcatheter heart valve prosthesis 200.

The capsule extension 110 with the blocking ring 130 acts in the same fashion as the capsule extension 110 with the blocking arms 125 described above with respect to FIGS. 3A-3D. Therefore, the details of the stages of deployment will not be repeated here and are incorporated into the embodiment of FIG. 4. It is understood that although a full ring is shown as the blocking ring 130, this is not meant to be limiting, and a partial ring may be used provided that the partial ring covers the access cells 285, as described above.

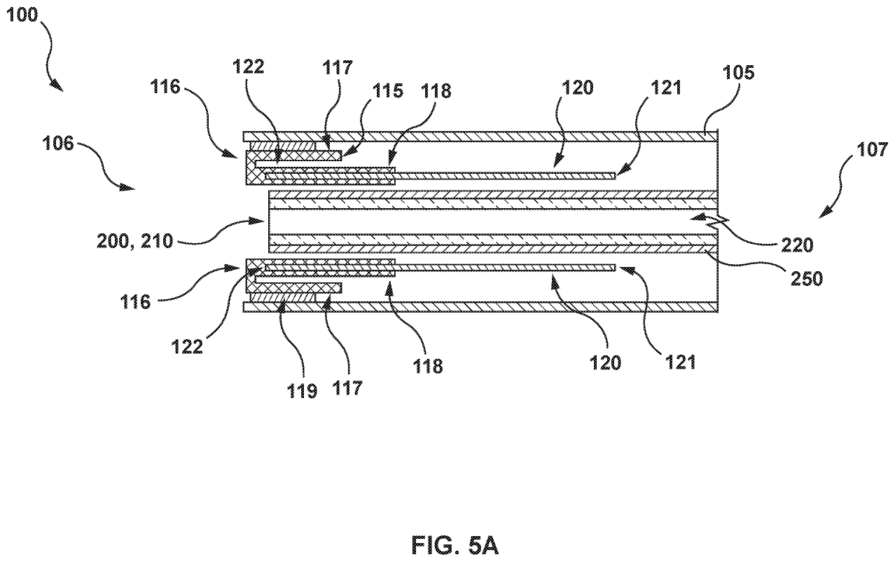

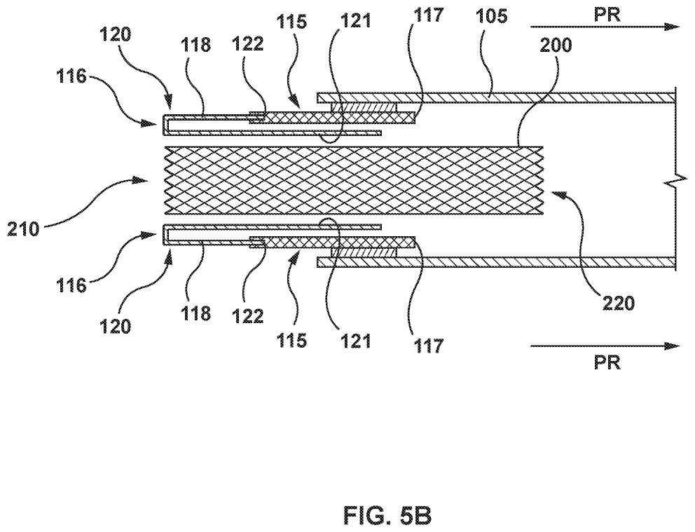

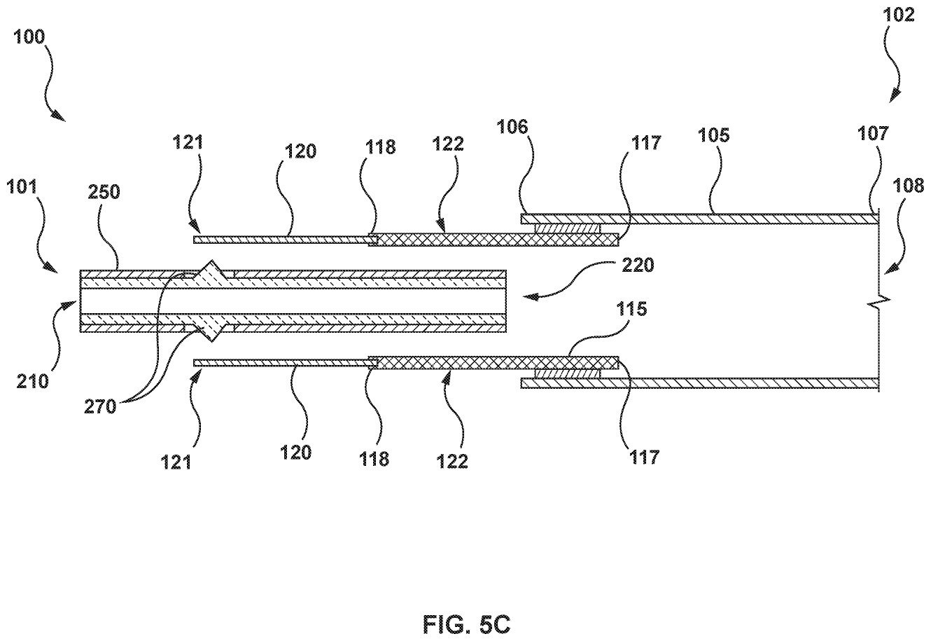

FIGS. 5A-5C show the delivery assembly 100 including a capsule extension 110 according to another embodiment hereof. The parts shown in FIG. 5A-5C are essentially the same as those should in FIGS. 3A-3D but are arranged differently with respect to the capsule 105. The configurations of FIGS. 5A-5C can be used with either the embodiment of FIGS. 3A-3D or the embodiment of FIG. 4.

FIG. 5A shows the delivery configuration in which the entire capsule extension 110 is loaded within the central lumen 108 of the capsule 105. The transcatheter heart valve prosthesis 200 is loaded within the central lumen 108 of the delivery capsule 105 in the crimped configuration and is disposed radially inward in relation to the capsule extension 110. As shown in FIG. 5A, the inflow end 210 of the frame 250 is adjacent the distal end 106 of the capsule 105 and the outflow end 220 of the frame 250 is disposed near the proximal end 107 of the capsule 105. As can be seen, the first end 117 of the mesh layer 115 is coupled to the interior surface of the distal end 106 of the capsule 105 at the bond 119. However, as opposed to the embodiment of FIGS. 3A-3B, the mesh layer 115 slightly extends distally from the bond 119 towards the distal end 106 of the capsule 105 until it reaches the fold 116. Thus, instead of the mesh layer 115 extending proximally to the fold 116 such that the fold 116 in FIG. 3A is proximal of the bond 119, the mesh layer 115 extends distally to the fold 116 such that the fold 116 is distal of the bond 119 in FIG. 5A. At the fold 116, the mesh layer 115 folds radially inward, towards the crimped transcatheter heart valve prosthesis 200, and extends back proximally towards the proximal end 107 of the capsule 105, as shown in FIG. 5A. In this configuration, the second end 118 of the mesh layer 115 terminates proximal to the first end 117 of the mesh layer 115 and is disposed proximal to the fold portion 116. Additionally, the second end 118 of the mesh layer 115 is disposed radially inward in relation to the first end 117 of the mesh layer 115. In this embodiment, the first ends 123 of the reaching arms 121 are bonded to or embedded within the second end 118 of the mesh layer 115 and the reaching arms 121 extend proximally therefrom. In other words, the second ends 122 of the reaching arms 121 extend proximally towards the proximal end 107 of the capsule 105 and are disposed proximal to the first ends 123 of the reaching arms 121, as shown in FIG. 5A. In the delivery configuration, the reaching arms 121 are disposed radially inward in relation to the first end 117 of the mesh layer 115 and the capsule 105. The capsule extension 110 may include blocking arms 125 coupled to the second ends 122 of the reaching arms 121 or the blocking ring 130 coupled to the second ends 122 of the reaching arms 121.

To transition the delivery assembly 100 from the delivery configuration to the deployed configuration, the capsule 105 is retracted proximally in order to release, or uncover, the transcatheter heart valve prosthesis 200 and the capsule extension 110. As shown in FIG. 5B, the capsule 105 is retracted in the direction of the arrows PR. As the capsule 105 is retracted, the first end 117 of the mesh layer 115 is also proximally retracted. However, the transcatheter heart valve prosthesis 200 does not move, but the transcatheter heart valve prosthesis 200 location relative to the capsule 105 and the mesh layer 115 is changed due to the movement of the capsule 105. The location of the fold 116 remains at the inflow end 210 of the transcatheter heart valve prosthesis 200, but the fold 116 is farther from the first end 117 of the mesh layer 115 due to movement of the capsule 105. This movement also causes the second ends 122 of the reaching arms 121 to move further distally relative to the transcatheter heart valve prosthesis 200, as shown in FIG. 5B. Thus, more of the capsule extension 110 is the outer layer in FIG. 5B than in FIG. 5A.

As the capsule 105 is further retracted proximally, the capsule extension 110 essentially inverts at the fold 116 such that the fold 116 is eliminated and the capsule extension 110 extends only distally from the bond 119, as shown in FIG. 5C. At this stage of the deployment, the capsule 105, the capsule extension 110, the reaching arms 120, and the blocking arms 125 (or the blocking ring 130) are in the same location as in FIGS. 3C and 3D. Thus, the blocking arms 125 (or the blocking ring 130) are aligned with the center portion of the access cells 285, as show in FIG. 5C, such as to prevent the leaflets 270 from extending through the access cells 285, as described above. Further, although not shown in FIG. 5C, at this stage of deployment, the inflow end 210 of the transcatheter heart valve prosthesis 200 will be at least partially deployed, as shown in FIG. 3D. Further, as explained above, this stage of deployment where the blocking arms 125 (or the blocking ring 130) are aligned with access cells 285 can be configured to occur at a desirable stage of deployment as described above, such as 0% to 70% deployment, or 20% to 50% deployment or 30% to 40% deployment. Deployment of the transcatheter heart valve prosthesis 200 may be completed by further retraction of the capsule 105, as described above.

It should be understood that various embodiments disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single device or component for purposes of clarity, it should be understood that the techniques of this disclosure may be performed by a combination of devices or components.

What is claimed is:

1. A delivery assembly for percutaneously delivering a transcatheter heart valve prosthesis into a vasculature of a patient, the delivery assembly comprising:
a capsule configured to constrain the transcatheter heart valve prosthesis therein; and
a capsule extension including:
a mesh layer coupled to the capsule;
reaching arms having a first end and a second end, the first end being coupled to the mesh layer; and
a blocking arm coupled the second ends of the reaching arms, wherein the blocking arm is configured to extend circumferentially around at least a portion of the transcatheter heart valve prosthesis to minimize leaflet protrusion through cells of a frame of the transcatheter heart valve prosthesis.

2. The delivery assembly of claim 1, wherein the blocking arm is a blocking ring configured to extend circumferentially around the entire circumference the transcatheter heart valve prosthesis.

3. The delivery assembly of claim 1, wherein the reaching arms comprise six reaching arms, wherein the blocking arm comprises three blocking arms, and wherein each blocking arm is coupled to the second ends of a corresponding adjacent pair of the reaching arms.

4. The delivery assembly of claim 1, wherein the mesh layer includes a first end coupled to a distal end of the capsule.

5. The delivery assembly of claim 4, wherein the delivery assembly includes a delivery configuration in which the capsule extension extends in a first direction from the first end of the mesh layer, folds inwardly at a fold, and extends from the fold in a second direction opposite the first direction.

6. The delivery assembly of claim 5, wherein the first direction is a proximal direction and the second direction is a distal direction.

7. The delivery assembly of claim 5, wherein the first direction is a distal direction and the second direction is a proximal direction.

8. The delivery assembly of claim 1, further comprising a transcatheter heart valve prosthesis, the transcatheter heart valve prosthesis including a frame and a valve structure coupled to the frame, wherein the valve structure includes a plurality of leaflets, and wherein the frame includes an access cell larger than other cells of the frame.

9. The delivery assembly of claim 8, wherein in a delivery configuration, the transcatheter heart valve prosthesis is disposed within the capsule and the capsule extension is disposed between the transcatheter heart valve prosthesis and the capsule.

10. The delivery assembly of claim 9, wherein in a deployed configuration of the capsule extension, the capsule extension extends distally from a distal end of the capsule, and the blocking arm extends circumferentially around the transcatheter heart valve prosthesis and across the access cell.

11. The delivery assembly of claim 10, wherein in the deployed configuration of the capsule extension, the transcatheter heart valve prosthesis is partially deployed from the capsule.

12. The delivery assembly of claim 11, wherein in the deployed configuration of the capsule extension, the transcatheter heart valve prosthesis is at 0% to 70% deployment, or 20% to 50% deployment, or 30% to 40% deployment.

13. A method of delivering and deploying a transcatheter heart valve prosthesis to a treatment site within a vasculature of a patient, the method comprising:
delivering the transcatheter heart valve prosthesis to the treatment site with the transcatheter heart valve prosthesis radially compressed within a capsule of a delivery device, the transcatheter heart valve prosthesis including a frame and a valve structure coupled to the frame, wherein the valve structure includes a plurality of leaflets, wherein the frame includes an access cell larger than other cells of the frame, and wherein the delivery device further includes a capsule extension coupled to the capsule and disposed between the transcatheter heart valve prosthesis and the capsule in a delivery configuration;
proximally retracting the capsule to release the transcatheter heart valve prosthesis from the capsule and transitioning the capsule extension to a deployed configuration of the capsule extension, wherein in the deployed configuration of the capsule extension, the capsule extension extends distally from a distal end of the capsule and surrounds a portion of the transcatheter heart valve prosthesis, and a blocking arm of the capsule extension extends circumferentially around at least a portion of the transcatheter heart valve prosthesis and across the access cell to minimize leaflet protrusion through the access cell;
determining whether to fully deploy the transcatheter heart valve prosthesis or to recapture the transcatheter heart valve prosthesis;
wherein if it is determined to recapture the transcatheter heart valve prosthesis, distally advancing the capsule to recapture the transcatheter heart valve prosthesis; and
wherein if it is determined to deploy the transcatheter heart valve prosthesis, proximally retracting the capsule further to fully deploy the transcatheter heart valve prosthesis.

14. The method of claim 13, wherein the blocking arm is a blocking ring, and wherein in the deployed configuration of the capsule extension, the blocking ring extends circumferentially around the entire circumference the transcatheter heart valve prosthesis.

15. The method of claim 13, wherein the capsule extension further includes a mesh layer coupled to the distal end of the capsule, reaching arms coupled at a first end thereof to the mesh layer and at a second end thereof to the blocking arm.

16. The method of claim 13, wherein in the delivery configuration, the capsule extension extends in a first direction from the first end of the mesh layer, folds inwardly at a fold, and extends from the fold in a second direction opposite the first direction.

17. The method of claim 16, wherein the first direction is a proximal direction and the second direction is a distal direction.

18. The method of claim 16, wherein the first direction is a distal direction and the second direction is a proximal direction.

19. The method of claim 13, wherein in the deployed configuration of the capsule extension, the transcatheter heart valve prosthesis is partially deployed from the capsule.

20. The method of claim 13, wherein in the deployed configuration of the capsule extension, the transcatheter heart valve prosthesis is at 0% to 70% deployment, or 20% to 50% deployment, or 30% to 40% deployment.
